(19) United States
(12) Patent Application Publication
(10) Pub. No.: US 2005/0170441 A1
(43) Pub. Date: Aug. 4, 2005

(54) Early detection of cancer of specific type using 1HNMR metabonomics

(75) Inventors: Kunle Odunsi, Williamsville, NY (US); Christine Ambrosone, Orchard Park, NY (US); James Alderfer, Williamsville, NY (US); JoAnne Alderfer, Williamsville, NY (US)

Correspondence Address: SIMPSON & SIMPSON, PLLC, 5555 MAIN STREET, WILLIAMSVILLE, NY, 14221-5406, US

(73) Assignee: Health Research, Inc., Buffalo, NY (US)

(21) Appl. No.: 10/937,948

(22) Filed: Sep. 10, 2004

Related U.S. Application Data
(60) Provisional application No. 60/502,794, filed on Sep. 12, 2003.

(51) Int. Cl. G01N 33/574; G06F 19/00; G01N 33/48; G01N 33/50

(52) U.S. Cl. 435/7.23; 436/64; 702/19

(57) ABSTRACT

A method for determining whether a patient has a particular type of cancer which comprises: a. obtaining an ascites, abnormal cell cellular fluid or serum sample from the patient;
b. diluting the sample with D₂O;
c. subjecting the sample to ¹HNMR to obtain a series of free induction decay outputs (FID's);
d. mathematically modifying the data to obtain ¹HNMR spectra;
e. correcting the ¹HNMR spectra for phase and baseline distortions;
f. data reducing the corrected ¹HNMR spectra to obtain a plurality of integral spectral segments; g. compensating for effects of variation in suppression of water resonance;
h. normalizing the resulting data to total spectral area to obtain normalized ¹HNMR spectra;
i. subjecting the normalized ¹HNMR spectra to principal component analysis to obtain normalized data; and j. plotting and comparing the normalized data with corresponding control data indicating the presence of the particular type of cancer to determine whether the sample indicates that the patient has the particular type of cancer.

### BACKGROUND OF THE INVENTION

This application claims the benefit under 35 U.S.C. §119(e) of U.S. Provisional Application No. 60/502,794, filed Sep. 12, 2003.

The present invention relates to early detection of cancer of a specific type such as ovarian, breast, prostate, colon, lung, pancreas and liver cancers. It is generally recognized that early detection of cancer and early detection of cancer type lend to better prognosis.

Unfortunately to date, early detection techniques are not as reliable as desired and for many cancer types there is no reliable early detection test at all. Further for most cancers there has been no single test that provides both reliable early detection of cancer as well as the type of cancer.

As an example, the link between stage and mortality suggests that early detection may have a significant impact on disease morbidity and mortality in epithelial ovarian cancer (EOC). At present there is no effective early detection strategy for ovarian cancer.

Epithelial ovarian cancer (EOC) is the leading cause of death from gynecologic malignancies. There are more than 23,000 cases annually in the United States, and 14,000 women can be expected to die from the disease (Greenlee, R. T., Hill-Harmon, M. B., Murray, T., and Thun, M. Cancer Statistics, 2001. CA Cancer J. Clin., 51: 15-36, 2001) in 2003. Despite modest improvements seen in response rates, progression-free survival and median survival using adjuvant platinum and paclitaxel chemotherapy following cytoreductive surgery, overall survival rates remain disappointing for patients with advanced EOC and primary peritoneal carcinomas (McGuire, W. P., Hoskins, W. J., Brady, M. F., Kucera, P. R., Partridge, E. E., Look, K. Y., Clarke-Pearson, D. L., and Davidson, M. Cyclophosphamide and Cisplatin Versus Paclitaxel and Cisplatin: A Phase III Randomized Trial in Patients with Suboptimal Stage III/IV Ovarian Cancer (from the Gynecologic Oncology. Group). Semin Oncol., 23: 40-47, 1996). This has been attributed to several reasons. First, in contrast to most other solid tumors, more than 75% of EOC patients are first diagnosed with advanced stage disease (FIGO III or IV). Whereas the small proportion of patients with accurately diagnosed stage I disease have 5 year survival rates in excess of 90% (Young, R. C., Walton, L. A., Ellenberg, S. S., Homesley, H. D., Wilbanks, G. D., Decker, D. G., Miller, A., Park, R. and Major, F., Jr. Adjuvant Therapy in Stage I and Stage II Epithelial Ovarian Cancer. Results of Two Prospective Randomized Trials. N. Engl. J. Med., 322: 1021-1027, 1990) the survival rate for women diagnosed with distant disease is only 25%. Secondly, although most patients with advanced disease initially respond to platinum and paclitaxel based chemotherapy including complete responses, the relapse rate is approximately 85% (Greenlee, R. T., Hill-Harmon, M. B., Murray, T., and Thun, M. Cancer Statistics, 2001. CA Cancer J. Clin., 51: 15-36, 2001). Within 2 years of cytoreductive surgery and systemic chemotherapy, tumors usually recur and once relapse occurs, there is no known curative therapy. Therefore, the link between stage and mortality suggests that early detection may have a significant impact on disease morbidity and mortality in EOC. The need for early detection is especially acute in women who have a high risk of ovarian cancer due to family or personal history of cancer, and for women with a genetic predisposition to cancer due to abnormalities in predisposition genes such as BRCA1 and BRCA2.

Although a number of potential early detection strategies have been studied in EOC (Menon, U. and Jacobs, I. J. Recent Developments in Ovarian Cancer Screening. Curr. Opin. Obstet. Gynecol., 12: 3942, 2000), these have shown only limited promise. The ideal test for the early detection of EOC should be non-invasive, acceptable to the screened population, with high validity, and at relatively low cost. In this regard, the application of novel approaches such as functional genomics, proteomics and metabonomics may substantially improve the ability to detect EOC at an early stage, leading to reduction in morbidity and mortality from the disease.

The majority of patients with EOC come from “low-risk” families. Current candidate strategies for early detection of EOC in this population are based on biochemical tumor markers evaluated mainly in the blood and biophysical markers assessed by ultrasound and/or Doppler imaging of the ovaries. The only biomarker that has been extensively studied for possible use in the early detection of EOC is CA125, a high-molecular-weight glycoprotein of unknown function (Fures, R., Bukovic, D., Hodek, B., Klaric, B., Herman, R., and Grubisic, G. Preoperative Tumor Marker CA125 Levels in Relation to Epithelial Ovarian Cancer Stage. Coll. Antropol., 23: 189-194, 1999; and Dorum, A., Kristensen, G. B., Abeler, V. M., Trope, C. G., and Moller, P., Early Detection of Familial Ovarian Cancer, Eur. J. Cancer, 32A: 1645-1651, 1996). A recent systematic review of the performance of the multimodal strategies of CA125 and ultrasound indicated that approximately 50% (95% confidence interval, CI: 23, 77) and 75% (95% CI: 35, 97) of patients were diagnosed at Stage I in CA125-based and ultrasound screening studies, respectively (Reviews, E. Screening for Ovarian Cancer. Database of Abstracts of Reviews of Effectiveness, Issue 1 Edition, Vol. 2003: Database of Abstracts of Reviews of Effects NHS Centre for Reviews and Dissemination, 2003). Unfortunately, the positive predictive values (PPV) of these strategies for the early detection of EOC using these modalities have been consistently less than 10% (Reviews, E. Screening for Ovarian Cancer. Database of Abstracts of Reviews of Effectiveness, Issue 1 Edition, Vol. 2003: Database of Abstracts of Reviews of Effects NHS Centre for Reviews and Dissemination, 2003; and van Nagell, J. R., Jr., DePriest, P. D., Reedy, M. B., Gallion, H. H., Ueland, F. R., Pavlik, E. J., and Kryscio, R. J., The Efficacy of Transvaginal Sonographic Screening in Asymptomatic Women at Risk for Ovarian Cancer. Gynecol. Oncol., 77: 350-356, 2000). Attempts to improve the PPV of these early detection strategies in EOC have met with limited success. These include the utilization of complex longitudinal algorithms for CA125 (Skates, S. J., Xu, F. J., Yu, Y. H., Sjovall, K., Einhom, N., Chang, Y., Bast, R. C., Jr., and Knapp, R. C. Toward an Optimal Algorithm for Ovarian Cancer Screening with Longitudinal Tumor Markers. Cancer, 76: 2004-2010, 1995; Zhang, Z., Bamhill, S. D., Zhang, H., Xu, F., Yu, Y., Jacobs, I., Woolas, R. P., Berchuck, A., Madyastha, K. R, and Bast, R. C., Jr. Combination of Multiple Serum Markers Using an Artificial Neural Network to Improve Specificity in Discriminating Malignant from Benign Pelvic Masses. Gynecol. Oncol., 73: 56-61, 1999; and McIntosh, M. W., Urban, N., and Karlan, B. Generating Longitudinal Screening Algorithms Using Novel Biomarkers for Disease. Cancer Epidemiol Biomarkers Prev., 11: 159-166, 2002), sequential testing (Berek, J. S. and Bast, R. C., Jr. Ovarian Cancer Screening. The Use of Serial Complementary Tumor Markers to Improve Sensitivity and Specificity for Early Detection. Cancer, 76: 2092-2096, 1995; and Jacobs, I. J., Skates, S. J., MacDonald, N., Menon, U., Rosenthal, A. N., Davies, A. P., Woolas, R., Jeyarajah, A. R., Sibley, K., Lowe, D. G., and Oram, D. H., Screening for Ovarian Cancer: A Pilot Randomised Controlled Trial. Lancet, 353: 1207-1210, 1999) and the addition of newer markers such as OVX-1 (Bast, R. C., Jr., Boyer, C. M., Xu, F. J., Wiener, J., Dabel, R., Woolas, R., Jacobs, I., and Berchuck, A. Molecular approaches to Prevention and Detection of Epithelial Ovarian Cancer. J. Cell. Biochem. Suppl., 23: 219-222, 1995), M-CSF (Suzuki, M., Ohwada, M., Aida, I., Tamada, T., Hanamura, T., and Nagatomo, M., Macrophage Colony-Stimulating Factor as a Tumor Marker for Epithelial Ovarian Cancer. Obstet. Gynecol., 82: 946-950, 1993), lysophosphatidic acid (Xu, Y., Shen, Z., Wiper, D., Wu, M., Morton, R., Elson, P., Kennedy, A. W., Bellinson, J., Markman, M., and Casey, G. Lysophosphatidic Acid as a Potential Biomarker for Ovarian and other Gynecologic Cancers. JAMA, 280: 719-723, 1998) and osteopontin (Kim, J. H., Skates, S. J., Uede, T., Wong Kk, K. K., Schorge, J. O., Feltmate, C. M., Berkowitz, R. S., Cramer, D. W., and Mok, S. C. Osteopontin as a Potential Diagnostic Biomarker for Ovarian Cancer. JAMA, 287: 1671-1679, 2002). In light of these considerations, novel approaches are needed for the early detection of EOC.

A recent study suggesting successful diagnosis of EOC using the proteomic approach underlines the considerable promise of new technologies for the detection of early-stage ovarian cancer (Petricoin, E. F., Ardekani, A. M., Hitt, B. A., Levine, P. J., Fusaro, V. A., Steinberg, S. M., Mills, G. B., Simone, C., Fishman, D. A., Kohn, E. C., and Liotta, L. A. Use of Proteomic Patterns in Serum to Identify Ovarian Cancer. Lancet, 359: 572-577, 2002). In this approach, whole serum samples from ovarian cancer patients and health controls were screened by surface enhanced laser desorption ionization-mass spectrometry (SELDI-MS). A bioinformatics tool was used to identify proteomic patterns in serum that distinguish neoplastic from non-neoplastic disease within the ovary. The results yielded a sensitivity of 100% and specificity of 95%. Although promising, a re-calculation of the PPV of this approach was only 1% when applied to the general population and 9% when applied to a high risk population (Rockhill, B. Proteomic Patterns in Serum and Identification of Ovarian Cancer. Lancet, 360: 169, author reply 170-171, 2002; Pearl, D. C. Proteomic Patterns in Serum and Identification of Ovarian Cancer. Lancet, 360: 169-170; author reply 170-171, 2002; and Elwood, M. Proteomic Patterns in Serum and Identification of Ovarian Cancer. Lancet, 360: 170; author reply 170-171, 2002). Therefore, alternative and/or complimentary novel early detection strategies in EOC are needed to achieve the very high specificity that would result in an acceptable PPV.

The strongest risk factor for ovarian cancer is the presence of an inherited mutation in one of the tow ovarian cancer susceptibility genes, BRCA1 or BRCA2. It is estimated that more than 10% of women in North America with invasive ovarian cancer carry a BRCA1 or BRCA2 mutation (Berchuck, A., Heron, K. A., Camey, M. E., Lancaster, J. M., Fraser, E. G., Vinson, V. L., Deffenbaugh, A. M., Miron, A., Marks, J. R., Futreal, P. A., and Frank, T. S. Frequency of Germline and Somatic BRCA1 Mutations in Ovarian Cancer. Clin. Cancer Res., 4: 2433-2437, 1998; and Risch, H. A., McLaughlin, J. R., Cole, D. E., Rosen, B., Bradley, L., Kwan, E., Jack, E., Vesprini, D. J., Kuperstein, G., Abrahamson, J. L., Fan, I., Wong, B., and Narod, S. A., Prevalence and Penetrance of Germline BRCA1 and BRCA2 Mutations in a Population Series of 649 Women with Ovarian Cancer. Am. J. Hum. Genet., 68: 700-710, 2001). Other risk factors include a family history of ovarian cancer, a previous diagnosis of breast cancer, and Ashkenazi Jewish ethnicity (Struewing, J. P., Hartge, P., Wacholder, S., Baker, S. M., Berlin, M., McAdams, M., Timmerman, M. M., Brody, L. C. and Tucher, M. A. The Risk of Cancer Associated with Specific Mutations of BRCA1 and BRCA2 among Ashkenazi Jews. N. Engl. J. Med., 336: 1401-1408, 1997; and Moslehi, R., Chu, W., Karlan, B., Fishman, D., Risch, H., Fields, A., Smotkin, D., Ben-David, Y., Rosenblatt, J., Russo, D., Schwartz, P., Tung, N., Warner, E., Rosen, B., Friedman, J., Brunet, J. S. and Narod, S. A. BRCA1 and BRCA2 Mutation Analysis of 208 Ashkenazi Jewish Women with Ovarian Cancer. Am. J. Hum. Genet., 66: 1259-1272, 2000). Although the higher incidence of disease in these groups would suggest that early detection is likely to be beneficial, there are insufficient data available to assess performance characteristics or define optimal strategies for early detection of EOC in this population.

A novel and unique strategy that provides a coherent perspective of the complete metabolic response of organisms to pathophysiological insult or genetic modification has been termed “metabonomics”. Metabonomics is based on the use of NMR (and other spectroscopic methods) and multivariate statistics for biochemical data generation and interpretation. NMR spectroscopy is based on the behavior of atoms placed in a static external magnetic field. Atomic nuclei possessing a property known as spin that is not equal to zero can give rise to NMR signals. Nuclei possessing this property are ¹H, ¹³C, ¹⁵N and ³¹P. Since protons are present in almost all metabolites in body fluids, an ¹H-NMR spectrum allows the simultaneous detection and quantification of thousands of proton-containing, low-molecular weight species within a biological matrix, resulting in the generation of an endogenous profile that may be altered in disease to provide a characteristic “fingerprint” (Nicholson, J. K., Lindon, J. C., and Holmes, E. “Metabonomics”: Understanding the Metabolic Responses of Living Systems to Pathophysiological Stimuli via Multivariate Statistical Analysis of Biological NMR Spectroscopic Data. Xenobiotica, 29: 1181-1189, 1999; Lindon, J. C., Nicholson, J. K., and Everett, J. R. NMR Spectroscopy of Biofluids. Annu. Rep. NMR Spectrosc., 38: 1-88, 1999; Lindon, J. C., Nicholson, J. K., Holmes, E., and Everett, J. R., Metabonomics: Metabolic Processes Studied by NMR Spectroscopy of Biofluids. Concepts Magn. Reson., 12: 289-320, 2000; and Nicholson, J. K., Connelly, J., Lindon, J. C., and Holmes, E. Metabonomics: A Platform for Studying Drug Toxicity and Gene Function. Nat. Rev. Drug Discov., 1: 153-161, 2002). A range of novel NMR strategies has also been developed for structure elucidation of metabolites in biofluids.

NMR-based metabonomics can offer advantages in a clinical setting in that it can be carried out on standard preparations of serum, plasma or urine circumventing the need for specialist preparations of cellular RNA and protein required for genomics and proteomics, respectively (Lindon, J. C., Nicholson, J. K., Holmes, E., and Everett, J. R., Metabonomics: Metabolic Processes Studied by NMR Spectroscopy of Biofluids. Concepts Magn. Reson., 12: 289-320, 2000; Nicholson, J. K. and Wilson, I. D. High Resolution Proton Magnetic Resonance Spectroscopy of Biological Fluids. Prog. Nucl. Magn. Reson. Spectrosc., 21: 449-501, 1989; Lindon, J. C., Holmes, E., and Nicholson, J. K., Pattern Recognition Methods and Applications in Biomedical Magnetic Resonance. Prog. Nucl. Magn. Reson. Spectrosc., 39: 1-40, 2001; and Holmes, E., Nicholson, J. K., and Tranter, G. Metabonomic Characterization of Genetic Variations in Toxicological and Metabolic Responses Using Probabilistic Neural Networks. Chem. Res. Toxicol., 14: 182-191, 2001) However, biological NMR spectra are extremely complex and much information can be lost even in rigorous statistical analysis of quantitative data as the essential diagnostic parameters are carried in the overall patterns of the spectra. Therefore, in order to reduce NMR data complexity and facilitate analysis data-reduction followed by chemometric methods such as principal components analysis (PCA) and partial least squares-discriminant analysis (PLS-DA), can be applied (Nicholson, J. K., Connelly, J., Lindon, J. C., and Holmes, E. Metabonomics: A Platform for Studying Drug Toxicity and Gene Function. Nat. Rev. Drug Discov., 1: 153-161, 2002). To further optimize the metabonomic approach, data filtering can be applied prior to chemometric analysis. One such filtering method, orthogonal signal correction (OSC) serves to remove variation within the NMR data that is not correlated to the focus of the study (Beckwith-Hall, B. M., Brindle, J. T., Barton, R. H., Coen, M., Holmes, E., Nicholson, J. K., and Antti, H. Application of Orthogonal Signal Correction to Minimize the Effects of Physical and Biological Variation in High Resolution ¹H NMR Spectra of Biofluids. Analyst, 127: 1283-1288, 2002). This data filtering is particularly pertinent to human metabonomic studies because of the immense variability in human populations compared to laboratory-controlled animal studies.

An integrated metabonomic approach has been applied to investigation of the presence and severity of coronary heart disease (CHD) (Brindle, J. T., Antti, H., Holmes, E., Tranter, G., Nicholson, J. K., Bethell, H. W., Clarke, S., Schofield, P. M., McKilligin, E., Mosedale, D. E., and Grainger, D. J. Rapid and Noninvasive Diagnosis of the Presence and Severity of Coronary Heart Disease Using ¹H-NMR-Based Metabonomics. Nat. Med., 8: 1439-1444, 2002). It was possible to completely separate CHD patients with stenosis of all three major arteries from subjects with normal coronary arteries using both unsupervised PCA and supervised PLS-DA applied to ¹H-NMR spectra of human serum (Brindle, J. T., Antti, H., Holmes, E., Tranter, G., Nicholson, J. K., Bethell, H. W., Clarke, S., Schofield, P. M., McKilligin, E., Mosedale, D. E., and Grainger, D. J. Rapid and Noninvasive Diagnosis of the Presence and Severity of Coronary Heart Disease Using ¹H-NMR-Based Metabonomics. Nat. Med., 8: 1439-1444, 2002). In another report, Brindle, et al. (Brindle, J. T., Nicholson, J. K., Schofield, P. M., Grainger, D. J., and Holmes, E. Application of Chemometrics to ¹H NMR Spectroscopic Data to Investigate a Relationship Between Human Serum Metabolic Profiles and Hypertension. Analyst, 128: 32-36, 2003) were able to distinguish serum samples from subjects with low/normal systolic blood pressure from borderline and high systolic blood pressures using NMR spectroscopy. These studies demonstrate the potential ability of ¹H-NMR based metabonomics to distinguish serum samples of individuals affected and unaffected by disease, without requiring preselection of measurable analytes.

### BRIEF DESCRIPTION OF THE INVENTION

In accordance with the invention, there is now provided a method for early detection of both cancer and cancer type including cancers for which reliable early cancer detection was not available.

In particular the method includes the following steps:
a. obtaining an ascites, abnormal cell cellular fluid or serum sample from the patient;
b. diluting the sample with D₂O;
c. subjecting the sample to ¹HNMR to obtain a series of free induction decay outputs (FID's);
d. mathematically modifying the data to obtain ¹HNMR spectra;
e. correcting the ¹HNMR spectra for phase and baseline distortions;
f. data reducing the corrected ¹HNMR spectra to obtain a plurality of integral spectral segments;
g. compensating for effects of variation in suppression of water resonance;
h. normalizing the resulting data to total spectral area to obtain normalized ¹HNMR spectra;
i. subjecting the normalized ¹HNMR spectra to principal component analysis to obtain normalized data; and
j. plotting and comparing the normalized data with corresponding control data indicating the presence of the particular type of cancer to determine whether the sample indicates that the patient has the particular type of cancer.

### BRIEF DESCRIPTION OF THE DRAWINGS

FIG. 1 is a PCA plot showing clear separation achieved between EOC serum samples (circles indicated by arrows) and healthy pre-menopausal controls (squares). Cancer in general is shown by a diamond.

FIG. 2 is a PCA plot showing clear separation achieved between EOC serum samples (circles indicated by arrows) and healthy post-menopausal controls (squares). Optimum separation occurs with respect to the second principal component graphed. Cancer in general is shown by a diamond.

FIG. 3 is a Cooman's plot demonstrating that the EOC sera class (diamond) and post-menopausal control sera class (circles) do not share multivariant space. Cancer in general is shown by a star.

FIG. 4 is a scatter plot and ROC analysis of H-NMR metabonomic profile of sera from healthy post-menopausal controls (solid circles) and EOC patients (empty circles).

### DETAILED DESCRIPTION OF THE INVENTION

Since cancer is now known to be a product of the tumor-host microenvironment (Liotta, L. A., and Kohn, E. C. The Microenvironment of the Tumor-Host Interface. Nature, 411: 375-379, 2001), the organ-specific milieu can generate, and enzymatically modify, multiple proteins, peptides, metabolites, and cleavage products at much high concentrations than for molecules derived only from the tumor cells. These metabonomic approaches therefore allow the elucidation of the molecules responsible for the different NMR spectral patterns of EOC patients as compared with normal subjects, leading to the identification of a panel of specific biomarkers and/or targets for therapeutic intervention. These approaches include both one-dimensional (1D) and multi-dimensional (2D) NMR experiments: i) NOESYPR1D provide 1D spectra with elimination of the large residual solvent (H₂O) resonance; ii) Carr-Purcell-Meiboom-Gill (CPMG) 1D spectra edit signals from large molecules out of a spectrum, leaving only signals from small molecule analytes; iii) COSY (2D) spectra show through-bond (usually 2-4 bonds) covalent connectivity, enabling assignment of 1D and 2D spectra resonances and construction of a molecular structure; iv) DOSY (2D) spectra analyze biofluids based on differences in molecular diffusion constants; v) NOESY and ROESY spectra provide through-space distances between hydrogen atoms, thereby enabling the construction (in combination with 1D and COSY experiments) of a 3D structure; and vi) HMQC and HSQC (2D) spectra facilitate correlating ¹H and ¹³C atoms of metabolites, thereby enabling metabolite identification (Robosky, L. C., Robertson, D. G., Baker, J. D., Rane, S., and Reily, M. D. In Vivo Toxicity Screening Programs Using Metabonomics. Comb. Chem. High Throughput Screen, 5: 651-662, 2002).

In accordance with the invention, samples used are generally liquid samples from the patient being tested, which liquids are fluids likely to carry organic molecules characteristic of the presence of a cancer being screened. Such samples are generally serum but may also be ascites from the area of a suspected tumor or cellular fluid obtained by lysing cells of suspected tissue.

Once obtained, the sample is diluted with deuterium dioxide (D₂O) in preparation for ¹H-NMR. Such dilution is usually at a ratio of between about 1:4 and 1:8 of sample to D₂O with the ratio commonly being from about 1:5 to about 1:6.

The diluted sample is subjected to ¹HNMR to obtain a series of free induction decay outputs (FID's). Such induction decay outputs can for example be obtained from decays resulting from pulsing at about 600 MHz using the pulse sequence RD-90°-t₁-90°-tₘ-90° where RD is a relaxation delay of 1.5 seconds during which water resonance is irradiated, t₁ is a fixed interval of 4 μs, and tₘ is a mixing time of 100 μs during which water is irradiated a second time. Multiple and sufficient FID data points can thus be obtained at varying spectral width intervals, e.g. 12.2 KHz at an acquisition time of 2.69 seconds, to enable the formation of decay spectra.

The data is mathematically modified to obtain NMR spectra. In performing such mathematical modification, the FIDs may be multiplied by an exponential weighting factor to modify line width, e.g. by a line broadening of 0.25 Hz. The FIDs (broadened or otherwise) are subjected to mathematical treatment to obtain an H¹NMR decay spectrum, the most common procedure being Fourier transformation.

The resulting NMR spectra are then corrected for phase and baseline distortions by comparison of phase and baseline with a standard, e.g. lactate CH₃δ1.33, and modifying the spectra so that the phase and baseline and resultant proportional spectral information are consistent with the standard.

The corrected ¹HNMR spectra are then data reduced to obtain a plurality of integral spectral segments of equal length, e.g. 200-250 segments at a length of δ0.04.
effects of variation in suppression of water resonance are compensated for by setting the region of water resonance (δ5.5 to δ4.75) to zero;
The resulting data is then normalized to total spectral area to obtain normalized ¹HNMR spectra,

The normalized ¹HNMR spectra is then subjected to principal component analysis and plotted and compared with corresponding control data indicating the presence of the particular type of cancer to determine whether the sample indicates that the patient has the particular type of cancer.

Up to now, no definitive screening test for early stage epidermal ovarian cancer (EOC) has been developed (38). The method of the invention has now been found to permit such a test.

In an effort to determine whether ¹H-NMR-based metabonomic analysis could identify EOC patients, the pre-operative serum samples of 38 patients with EOC and 53 normal health women (controls) were collected under an approved IRB protocol. The stage distribution of the patients were as follows: Stage I: 2 patients; Stage IIIC: 34 patients; Stage 1V: 2 patients. Among patients with advanced disease (Stages IIIC and IV), 4 (11%) had normal pre-operative serum CA125 levels (<35 units/ml). In addition, pre-operative CA125 was normal in 1 of the tow patients with stage I disease. The age range of the study patients was 46-86 years. Twenty-one of the control subjects were pre-menopausal (age range 22-44 years) while the remaining 32 subjects were postmenopausal (age range 45-75 years). Aliquots of serum were stored at −80° C. until assayed.

Samples (100 μl) were diluted with solvent solution (99.9% D₂O) (55 μl) in 5-mm precision NMR tubes (Norell, Inc., Landisville, N.J. USA). Conventional ¹H-NMR spectra of the serum samples were measured at 600.22 MHz on a Bruker AMX-600 spectrometer (Billerica, Mass.) operating at 600 MHz ¹H frequency, using the pulse sequence: RD-90°-acquire free induction decay (FID) (i.e., the NOESYPRID pulse sequence). RD represents a relaxation delay of 1.5s during which the water resonance is selectively irradiated, and to corresponds to a fixed interval of 4 μs. The water resonance is irradiated for a second time during the mixing time ((tₘ, 100 ms). For each sample, 128 FIDs were collected into 64K data points using a spectral width of 12.2 KHz and an acquisition time of 2.69s. The FIDs were multiplied by an exponential weighting function corresponding to a line broadening of 0.25 Hz before Fourier transformation. The acquired NMR spectra were corrected for phase and baseline distortions using UXNMR (version 97) and referenced to lactate (CH₃δ1.33).

The ¹H-NMR spectra (δ10-0.2) were automatically data-reduced to 200-250 integral segments of equal length (δ0.04) using NutsPro (version 20021122, Acorn NMR, Inc.). Each segment consisted of the integral of the NMR region to which it was associated. To remove the effects of variation in the suppression of the water resonance, the region δ5.5 to 4.75 was set to zero integral. The data were normalized to total spectral area and centered scaling was applied.

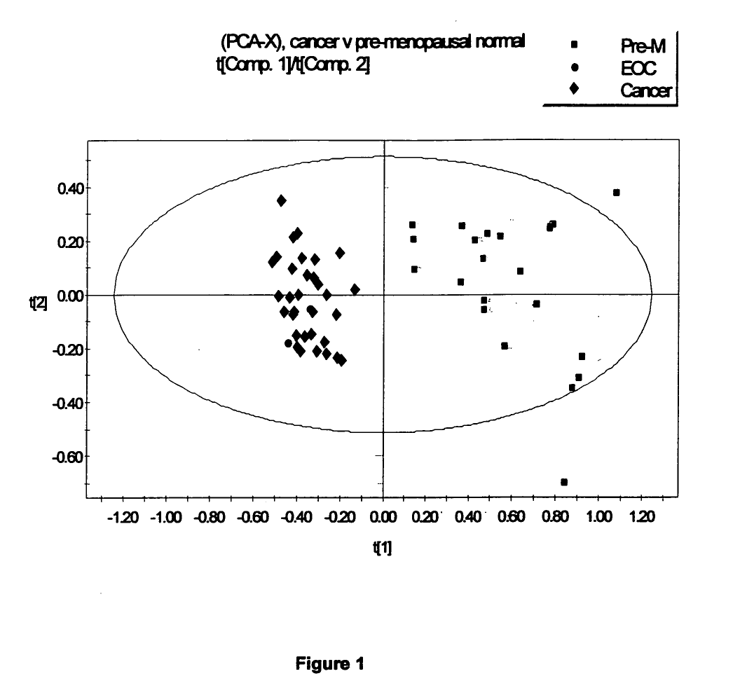

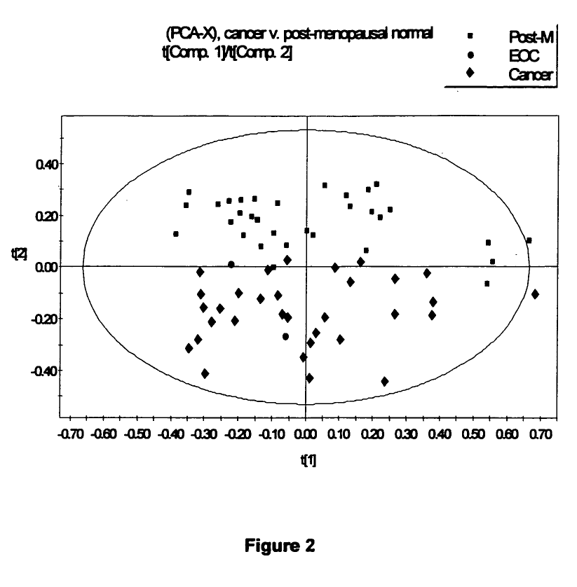

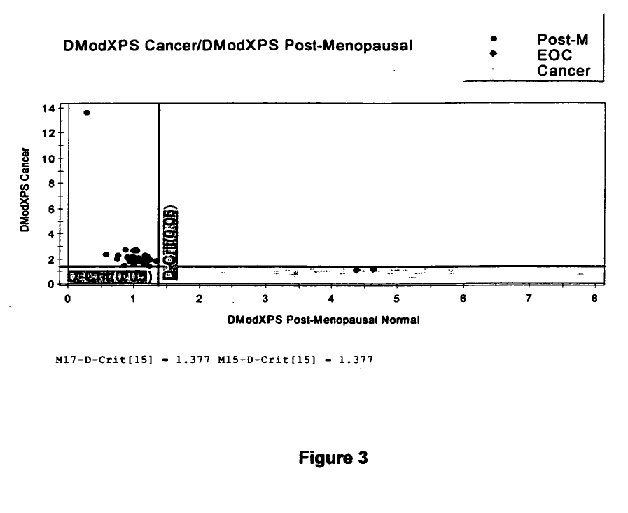

Principal component analysis (PCA) is an unsupervised method (i.e. analysis performed without use of knowledge of the sample class) that reduces the dimensionality of the data input whilst expressing much of the original n-dimensional variance in a 2 or 3-D map (Eriksson, L., Johansson, E., Kettaneh-Wold, N., and Wold, S. Introduction to Multi-and Megavariate Data Analysis Using Projection Methods (PCA & OLS). Umea, Sweden: Umetrics, 1999). Prior to PCA analysis, all NMR data were mean-centered and pareto-scaled (Wold, S., Antti, H., Lindgren, F., and Ohman, J. Orthogonal Signal Correction of Near-Infrared Spectra. Chemom. Intell. Lab. Syst., 44: 175-185, 1998) to give each variable a variance numerically equal to its standard deviation. PCA was carried out on the ¹H-NMR data from the sera of EOC patients and controls to plot data in order to indicate relationships between samples in the multidimensional space. The principal components were displayed as a set of “scores” (t), which highlight clustering or outliers, and a set of “loadings” (p), which highlight the influence of input variables on t. This dataset of NMR spectra displayed good discrimination between EOC patients and controls. Thus, we were able to correctly separate all of the 38 cancer specimens (100%) and all of the 21 pre-menopausal normal samples (100%) as shown in FIG. 1. In addition, it was possible to correctly separate 37 of 38 (97.4%) cancer specimens and 31 of 32 (97%) postmenopausal control serum specimens, as shown in FIG. 2. A Cooman's plot of the data (Coomans, D., Broeckaert, I., Derde, M. P., Tassin, A., Massart, D. L., and Wold, S. Use of a Microcomputer for the Definition of Multivariate Confidence Regions in Medical Diagnosis Based on Clinical Laboratory Profiles. Comput. Biomed. Res., 17: 1-14, 1984), which plots class distances against each other, demonstrates that the EOC sera class and the postmenopausal control sera class did not share multivariate space, providing validation for the class separation as shown in FIG. 3. Therefore, it should be possible to predict whether future samples can be classified as cancer, healthy postmenopausal, or neither. This preliminary data demonstrated that ¹H-NMR-based metabonomic analysis of serum samples could achieve a clinically useful performance for the identification of serum samples of patients with EOC.

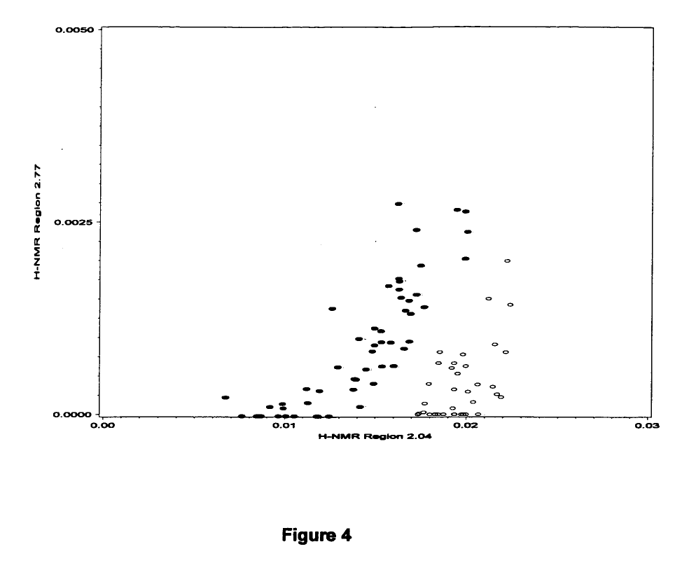

Univariate ROC analyses were carried out via individual logistic regressions for each of 219 ¹H-NMR regions in order to examine their utility for predicting EOC. The sensitivity and specificity trade-offs were summarized for each variable using the area under the ROC curve denoted AUC, and calculated using the trapezoidal rule. An AUC value of 1.0 corresponds to a prediction model with 100% sensitivity and 100% specificity, while an AUC value 0.5 corresponds to a poor predictive model (see Pepe, M. S. A Regression Modeling Framework for Receiver Operating Characteristic Curves in Medical Diagnostic Testing. Biometrika, 84: 595-608, 1997 for an overview of ROC analyses via logistic regression modeling). The best tow variable models were then fit starting from the univariate information via a forward stepwise selection using the AUC as the criteria for a variable's entry into the model. The data showed that a tow variable model consisting of ¹H-NMR regions 2.77 μs from the origin and 2.04 μs from the origin provided a perfect fitting model, i.e. AUC=1.0. A scatterplot is provided in FIG. 4, which clearly illustrates the delineation between the two groups. Of note, the univariate model that considered only region 2.04 μs gave an AUC=0.942 while the AUC for the univariate model for region 2.77 μs and AUC=0.689, i.e. prediction based upon region 2.04 is enhanced conditional upon the information contained in region 2.77 μs. We hypothesize that the preliminary information that we have derived from this ROC analysis will allow us to refine this model for early stage EOC, and that this approach could represent a novel strategy for the early detection of EOC.

Based on the promising results showing complete separation of patients with EOC and controls using unsupervised PCA and ROC analysis applied to ¹H-NMR spectra of sera, we have proceeded to identify the molecules responsible for the differences in spectral patterns utilizing a previously described methodology (Gavaghan, C. L., Holmes, E., Lenz, E., Wilson, I. D., and Nicholson, J. K., An NMR-Based Metabonomic Approach to Investigate the Biochemical Consequences of Genetic Strain Differences: Application to the C57BL10J and Alpk:ApfCD Mouse. FEBS Lett., 484: 169-174, 2000). Our preliminary observations suggest greater amount of 3-hydroxybutyrate and isobutyrate in the sera of EOC patients compared with postmenopausal controls (data not shown). The biological significance of these observations is currently unclear.

¹H-NMR metabonomic analysis has been done on serum samples as described above, obtained from women with EOC and health pre- and post-menopausal controls. The resulting data indicates that the sera from patients with and without disease can be identified with 100% sensitivity and specificity at the ¹H-NMR regions 2.77 μs and 2.04 μs from the origin (AUC of ROC curve=1.0). In addition, we have identified some of the variables responsible for differences in spectral patterns between EOC patients and health controls. In accordance with the invention, ¹H-NMR metabonomic analysis of sera is a useful strategy for the detection of early EOC.

1. A method for determining whether a patient has a particular type of cancer which comprises:
a. obtaining an ascites, abnormal cell cellular fluid or serum sample from the patient;
b. diluting the sample with D₂O;
c. subjecting the sample to ¹HNMR to obtain a series of free induction decay outputs (FID's);
d. mathematically modifying the data to obtain ¹HNMR spectra;
e. correcting the ¹HNMR spectra for phase and baseline distortions;
f. data reducing the corrected ¹HNMR spectra to obtain a plurality of integral spectral segments;
g. compensating for effects of variation in suppression of water resonance;
h. normalizing the resulting data to total spectral area to obtain normalized ¹HNMR spectra;
i. subjecting the normalized 1HNMR spectra to principal component analysis to obtain normalized data; and
j. plotting and comparing the normalized data with corresponding control data indicating the presence of the particular type of cancer to determine whether the sample indicates that the patient has the particular type of cancer.

2. The method of claim 1 where, in step d., the data is mathematically modified subjecting the FID data to Fourier transformation.

3. The method of claim 2 where prior to Fourier transformation the FID data is mathematically weighted to obtain weighted FID data for the Fourier transformation.

4. The method of claim 1 wherein the particular type of cancer is ovarian cancer.

5. The method of claim 3 wherein the particular type of cancer is ovarian cancer.

6. The method of claim 1 wherein the particular type of cancer is breast cancer.

7. The method of claim 3 wherein the particular type of cancer is breast cancer.

8. The method of claim 1 wherein the particular type of cancer is prostate cancer.

9. The method of claim 3 wherein the particular type of cancer is prostate cancer.

10. The method of claim 1 wherein the particular type of cancer is colon cancer.

11. The method of claim 3 wherein the particular type of cancer is colon cancer.

12. The method of claim 1 wherein the particular type of cancer is lung cancer.

13. The method of claim 3 wherein the particular type of cancer is lung cancer.

14. The method of claim 1 wherein the particular type of cancer is pancreatic cancer.

15. The method of claim 3 wherein the particular type of cancer is pancreatic cancer.

16. The method of claim 1 wherein the particular type of cancer is hepatic cancer.

17. The method of claim 3 wherein the particular type of cancer is hepatic cancer.

18. The method of claim 4 where peaks in the ¹HNMR regions 2.77 μs and 2.04 μs indicate the presence of ovarian cancer.

19. The method of claim 4 where peaks in the ¹HNMR regions 2.77 μs and 2.04 μs indicate the presence of ovarian cancer.
